(19) United States
(12) Patent Application Publication
(10) Pub. No.: US 2018/0369421 A1
(43) Pub. Date: Dec. 27, 2018

(54) METHOD FOR REDUCING THE PLACEBO EFFECT IN CLINICAL TRIALS

(71) Applicant: Leon I. Rosenberg, Cherry Hill, NJ (US)

(72) Inventor: Leon I. Rosenberg, Cherry Hill, NJ (US)

(21) Appl. No.: 16/017,254

(22) Filed: Jun. 25, 2018

Related U.S. Application Data
(60) Provisional application No. 62/525,491, filed on Jun. 27, 2017.

(51) Int. Cl. A61K 49/00; A61K 31/4168; A61K 31/155; A61K 31/135; A61K 36/185; A61K 31/137

(52) U.S. Cl. CPC A61K 49/00; A61K 31/4168; A61K 31/137; A61K 31/135; A61K 36/185; A61K 31/155

(57) ABSTRACT

Methods for lowering the placebo effect or inducing an Acebo effect in a clinical trial are disclosed. The methods are directed to including a group of subjects in a clinical trial wherein the additional group is administered a substance that has at least one known effect which can worsen the disease or disorder that is being studied in the clinical trial, or can cause one of the primary or secondary subsymptoms of the disease or disorder that is being studied in the clinical trial, or can cause a related or unrelated effect of the disease or disorder that is being studied in the clinical trial (an Acebo).

### CROSS-REFERENCE TO RELATED APPLICATION

The present invention claims the benefit of U.S. Provisional Patent Application No. 62/525,491 filed Jun. 27, 2017 entitled “Method for Reducing the Placebo Effect in Clinical Trials,” the entire disclosure of which is incorporated by reference herein for all purposes.

### BACKGROUND OF THE INVENTION

The present invention relates generally to the field of clinical trials for screening and identifying medically effective compounds. In particular, the present invention relates to a method for reducing the placebo effect in a clinical trial (“CT”).

Before a new drug or treatment can be marketed, it must be tested and approved for clinical efficacy and safety. Clinical trials used in drug development often involve comparison of a new treatment, new dose, existing treatment, existing doses, or a combination of these with a placebo. A placebo is an inert substance, usually made to look similar to the test substance, which has no therapeutic effect. Typically, these clinical trials are double-blinded, i.e., neither the clinical investigators nor the subjects know who is getting a placebo and who is getting the new or existing treatment.

Individuals receiving placebo treatments often experience an improvement in their condition, the “placebo effect,” or alternatively, can experience side effects similar to those exhibited by individuals receiving drug treatment, the “nocebo effect.” The placebo effect is common in medicine and clinical trials and has been increasing over the years. The more often in a given clinical trial that the placebo is able to demonstrate its ability to either treat, partially treat, be effective, be partially effective, or meet one or more of the designated study benchmarks or efficacy measures, etc., the more likely the study will not be positive, i.e., not show that the new treatment has a beneficial effect and is superior to standard treatment (or superior to placebo). The placebo effect has been shown to be as high as 61.3% where treatment effect was defined as a 75% or more reduction in symptoms. McElroy, S L et al., JAMA Psychiatry. 2015 March; 72 (3):235-46. doi: 10.1001/jamapsychiatry.2014.2162. The lower the placebo rate, the more likely it is that the study will be positive, i.e., the lower the placebo rate, the more likely it is that the study will demonstrate that the investigational product separates from placebo.

A statistically significant difference to be achieved between active drug/treatment/product and placebo is enumerated prior to the time that the clinical trial is conducted. In placebo-controlled studies, the higher the placebo effect, the more costly the clinical trial as more subjects need to be enrolled in the study to meet the enumerated statistically significant difference between drug and placebo. A lower placebo effect would increase the likelihood of achieving statistically significant separation of drug effect from placebo effect. A lower placebo effect would decrease the costs of conducting positive studies on efficacious drugs/treatments. A lower placebo effect will enable efficacious drugs to demonstrate their efficacy and therefore achieve governmental approval to be marketed to the public.

Clinical trials have attempted to use a variety of techniques to reduce the placebo response, including: the use of single blind placebo lead-in studies (letting the investigator and his/her team know that the subjects will be receiving, unbeknownst to the subjects, placebo at the beginning of the study to see who responds to the placebo, and then taking those individuals who respond to the placebo out of the study); IVRS (interactive voice response system)—an attempt to take the bias of raters, who in theory might rate too high or too low, i.e., for their own financial benefit, out of the statistics; remote centralized raters—another attempt to take the bias of raters to rate too high or too low, i.e., for their own financial benefit out of the statistics; study protocols in which both the subjects and investigators do not know when the subjects are randomized to potentially receive active medication, to remove the unconscious bias of subjects, raters and investigators; changing study end points, to remove the unconscious bias of subjects, raters and investigators; and the Sequential Comparison Design Techniques invented and patented by both Dr. Maurizio Fava, the Director of Clinical Research and Executive Vice Chair of Psychiatry at the Massachusetts General Hospital and David Schoenfeld, PhD of the Massachusetts General Hospital Biostatistics Center in which placebo non-responders are recycled in the study to prevent only using high placebo response rates in the statistical analysis of data. Although each of the above techniques may have shown actual or theoretical promise, all continue to result in too many negative clinical trials, despite efficacious investigational medications and investigational drugs.

There is a need in the art for novel methods of lowering the placebo effect to improve the accuracy and success of clinical trials and help bring effective medications and treatments to market. This invention fulfills this need.

### BRIEF SUMMARY

In accordance with an exemplary embodiment, the present invention provides a method for lowering a placebo effect in a clinical trial comprising administering a test substance to a participant in a first group of a plurality of treatment groups of study participants, administering a placebo to a participant in a second group of said plurality of treatment groups of study participants, administering a substance that has at least one known effect which can worsen the disease or disorder that is being studied in the clinical trial (an Acebo) to a participant in a third group of said plurality of treatment groups of study participants, wherein said participant in each of said treatment groups has a disease or disorder that is being studied, and wherein said participant in each of said treatment groups has been informed that he or she may be receiving a test substance, a placebo or an Acebo, (i.e., a substance that could make them worse, their disease state worse, their symptoms worse, etc.) determining whether each participant in each of said treatment groups is a responder, a partial responder or a non-responder; and comparing the percentage of responders, partial responders, or non-responders in the first (test) group to the percentage of responders, partial responders, or non-responders in the second (placebo) group. In another embodiment, the percentage of responders, partial responders, or non-responders in the first (test) group is compared to the percentage of responders, partial responders, or non-responders in the third (Acebo) group. In yet another embodiment, the percentage of responders, partial responders, or non-responders in the first (test) group is compared to the sum of the percentage of responders, partial responders, or non-responders in the second (placebo) group and the percentage of responders, partial responders, or non-responders in the third (Acebo) group. In one other embodiment, the sum of the percentage of responders, partial responders, or non-responders in the first (test) group and the percentage of responders, partial responders, or non-responders in the third (Acebo) group is compared to the percentage of responders, partial responders, or non-responders in the second (placebo) group.

In accordance with another exemplary embodiment, the present invention provides a method for inducing an Acebo effect in a clinical trial comprising administering a test substance to a first group of a plurality of treatment groups of study participants and administering a substance that has at least one known effect which can worsen the disease or disorder that is being studied in the clinical trial (an Acebo) to a second group of said plurality of treatment groups of study participants, wherein each participant in each of said treatment groups has been informed that he or she may be receiving a test substance or an Acebo, determining whether each participant in each of said treatment groups is a responder, a partial responder or a non-responder; and comparing the percentage of responders, partial responders or non-responders in the first (test) group to the percentage of responders, partial responders or non-responders in the second (Acebo) group.

In accordance with another exemplary embodiment, the present invention provides a method for lowering a placebo effect in a clinical trial comprising performing a first phase of testing, said first phase of testing including administering a test substance to a first group of a plurality of treatment groups of study participants, and administering a placebo to a remainder of said plurality of treatment groups of study participants, determining whether each participant in each of said treatment groups in said first phase is a responder, partial responder or a non-responder; comparing the percentage of responders in the first (test) group of said first phase to the percentage of responders in the second (placebo) group of said first phase, and performing a second phase of testing, said second phase of testing including administering a test substance to a first group of a plurality of treatment groups of study participants, administering a placebo to a second group of said plurality of treatment groups of study participants, administering a substance that has at least one known effect which can worsen the disease or disorder that is being studied in the clinical trial (an Acebo) to a third group of said plurality of treatment groups of study participants, wherein each participant in each of said treatment groups has been informed that he or she may be receiving a test substance, a placebo or an Acebo; determining whether each participant in each of said treatment groups in said second phase is a responder or a non-responder; comparing the percentage of responders in the first (test) group in said second phase to the percentage of responders in the second (placebo) group in said second phase, and analyzing data from said first phase of testing and data from said second phase of testing with a processor, wherein a greater difference between participants receiving the test substance and participants receiving the placebo in said second phase compared to the difference between participants receiving the test substance and participants receiving the placebo in said first phase indicates a lowering of said placebo effect.

In one embodiment, the Acebo is a U.S. Food and Drug Administration (FDA)-approved drug.

In one embodiment, the clinical trial is applied to any type of disease or disorder or condition. In another embodiment, the disease or disorder is selected from the group consisting of: anxiety, depression, mania, hypertension, hypotension, pain, inflammation, gastroesophageal reflux disease (GERD), Alzheimer's disease, diabetes, constipation, Parkinson's disease, gastrointestinal (GI) bleeding, gout and memory loss or dementia.

In one embodiment, the Acebo can increase mania (excessive elation and/or irritability). In one embodiment, the Acebo is selected from the group consisting of: corticosteroids (such as triamcinolone acetonide, fluticasone, hydrocortisone, prednisone, and triamcinolone), cyclosporine, carbidopa/levodopa, baclofen, antidepressants (for example, monoamine oxidase inhibitors (MAOIs) such as phenelzine and tranylcypromine; selective serotonin reuptake inhibitors (SSRIs) such as escitalopram, fluoxetine, and paroxetine; serotonin/norepinephrine reuptake inhibitors (SNRIs) such as desvenlafaxine, duloxetine, levomilnacipran, venlafaxine, and tricyclic antidepressants (such as nortriptyline)), methylphenidate, amphetamine, levothyroxine, antibiotics (such as ciproflozacin and gentamicin), antimalarial drugs (such as chloroquine and mefloquine), and antineoplastic drugs (such as 5-fluorouracil and ifosfamide).

In one embodiment, the Acebo can increase depression. In one embodiment, the Acebo is selected from the group consisting of: barbiturates (such as phenobarbital and secobarbital), benzodiazepines (such as alprazolam, clonazepam, chlordiazepoxide, diazepam, flurazepam, lorazepam, and triazolam), isotretinoin, hormones (for example, estrogens and progestogens such as etonogestrel, drospirenone, levonorgestrel, ethinyl estradiol, norethindrone, norgestimate, norgestrel, and desogestrel), corticosteroids, opioids (such as codeine, morphine, aspirin/oxycodone, meperidine, and oxycodone), anticholinergics, high blood pressure medications (for example, calcium-channel blockers such as diltiazem, nifedipine, and verapamil, beta-blockers such as atenolol, carvedilol, and metoprolol and alpha 2 agonists such as clonidine and guanfacine), interferon alpha, alcohol, anticonvulsants (such as ethosuximide and methsuximide), varenicline, acyclovir, Parkinson's medication, such as carbidopa-levodopa and carbidopa-levodopa-entacapone, dopamine agonists such as pramipexole and ropinorole, stimulants, anticonvulsants, proton pump inhibitors and H2 blockers, Anticholinergic drugs used to treat stomach cramps and other GI disorders, and statins (such as atorvastatin, fluvastatin, pravastatin, and simvastatin).

In one embodiment, the Acebo can increase hypertension. In one embodiment, the Acebo is selected from the group consisting of: acetaminophen, non-steroidal anti-inflammatory drugs (NSAIDs) (such as aspirin, meloxicam, ibuprofen and naproxen), indomethacin, piroxicam, decongestants (such as pseudoephedrine and phenylephrine), alcohol, amphetamines, 3,4-methylenedioxymethamphetamine (MDMA) and its derivatives, cocaine, caffeine, methylphenidate, antidepressants (such as venlafaxine, bupropion, desipramine, monoamine oxidase inhibitors, tricyclic antidepressants, and fluoxetine), corticosteroids, cyclosporine, tacrolimus, erythropoietin and herbal supplements (such as arnica (Arnica montana), bitter orange (Citrus aurantium), ephedra (ma-huang), ginkgo (Ginkgo bilboa), ginseng (Panax quinquefolius and Panax ginseng), guarana (Paullinia cupana), licorice (Glycyrrhiza glabra), senna (Cassia senna), and St. John's Wort (Hypericum perforatum)).

In one embodiment, the Acebo can increase anxiety. In one embodiment, the Acebo is selected from the group consisting of: asthma medicines (such as albuterol, salmeterol, and theophylline), blood pressure medicines (such as methyldopa), hormones (for example, estrogens and progestogens such as etonogestrel, drospirenone, levonorgestrel, ethinyl estradiol, norethindrone, norgestimate, norgestrel, and desogestrel), amphetamines and amphetamine derivatives, methylphenidate, atomoxetine, cocaine, caffeine, steroids (such as cortisone, dexamethasone, and prednisone), thyroid medicines (such as levothyroxine), decongestants (such as pseudoephedrine and phenylephrine), phenytoin, levodopa, quinidine, and antidepressants (such as bupropion, fluoxetine, venlafaxine, and selective serotonin reuptake inhibitors (SSRIs) such as escitalopram, fluoxetine, and paroxetine).

In one embodiment, the Acebo can worsen diabetes. In one embodiment, the Acebo is selected from the group consisting of: glucocorticoids, thiazide diuretics, beta-blockers, niacin, and antipsychotic drugs (such as olanzapine, clozapine, aripiprazole, chlorpromazine, flufenazine, haloperidol, molindone, perphenazine, perphenazine and amitriptyline, risperidone, thioridazine, thiothixene, and atypical antipsychotics such as quetiapine, cariprazine, brexpiprazole, ziprasidone, and asenapine).

In one embodiment, the Acebo can worsen GERD. In one embodiment, the Acebo is selected from the group consisting of: antibiotics (such as tetracycline), bisphosphonates (such as alendronate, ibandronate and risedronate), iron supplements, quinidine, pain relievers (such as ibuprofen, naproxen and aspirin), potassium supplements, anticholinergics (such as oxybutynin), tricyclic antidepressants (such as amitriptyline and doxepin), blood pressure medicines (for example, calcium channel blockers and beta blockers), narcotics (opioids) (such as codeine, morphine, oxycontin and hydrocodone/acetaminophen), progesterone, asthma medicines (such as theophylline), sedatives (for example, benzodiazepines such as diazepam and temazepam), antidepressants (such as imipramine and amitriptyline), and anticholinergics (such as oxybutynin and promethazine).

In one embodiment, the Acebo can cause or worsen constipation. In one embodiment, the Acebo is selected from the group consisting of: amitriptyline, doxepin HCl, imipramine, amitriptyline, trimipramine, doxepin, desipramine, nortriptyline, protriptyline, haloperidol, pianozide, risperidone, thiothixene, olanzapine, clozapine, chlorpromazine, thioridazine, calcium-channel blockers, clonidine, clonidine/chlorthalidone, disopyramide, bromocriptine, trihexyphenidyl, benztropine mesylate, biperiden, procyclidine, cholestyramine, sucralfate, ferrous gluconate, ferrous sulfate, hydrocodone bitartrate, chlorpheniramine polistirex, hydrocodone tartrate, homatropine methylbromide, sulindac, ketoprofen, loperamide hydrochloride, acetaminophen and codeine, fentanyl, hydrocodone, hydromorphone, meperidine, methadone, morphine/morphine sulfate, oxycodone, oxymorphone, propoxyphene, tramadol, and St. John's Wort.

In one embodiment, the Acebo can worsen movement symptoms of Parkinson's disease, including slowness, stiffness, tremor and dyskinesia. In one embodiment, the Acebo is selected from the group consisting of: aripiprazole, chlorpromazine, flufenazine, haloperidol, molindone, perphenazine, perphenazine and amitriptyline, risperidone, thioridazine, thiothixene, and atypical antipsychotics such as quetiapine, cariprazine, brexpiprazole, ziprasidone, and asenapine, as well as the anti-nausea medicines metoclopramide, phenothiazine, promethazine.

In one embodiment, the Acebo can worsen GI ulcers and GI bleeding. In one embodiment, the Acebo is selected from the group consisting of: diflunisal, ibuprofen, naproxen, flurbiprofen, diclofenac-misoprostol, aspirin diclofenac, cefuroxime, cinoxacin, sulindac, oxaprozin, ketorolac, ketorolac tromethamine, acetaminophen, acetaminophen-aspirin-caffeine, piroxicam, ketoprofen, indomethacin, etodolac, meclofenamate, meloxicam, nabumetone, fenoprofen, bisoprolol fumerate, piroxicam, mefenamic acid, tolmetin, and acetaminophen-naproxen.

In one embodiment, the Acebo can worsen gout pain. In one embodiment, the Acebo is selected from the group consisting of: aspirin and diuretics

In one embodiment, the Acebo can worsen cognitive symptoms. In one embodiment, the Acebo is selected from the group consisting of: those that block the effects of acetylcholine, such as tolteridine, some antidepressants (especially the tricyclics such as amitriptyline), antipsychotics, cardiac medications, antispasmodics, antivertigo medications, antiparkinsonian medications having anticholinergic effects, anxiety and insomnia medications, benzodiazepines, such as lorazepam or sleeping pills such as temazepam, corticosteroids, pain medications, and chemotherapy.

### DETAILED DESCRIPTION OF THE INVENTION

This invention is related to the unexpected discovery of a novel approach to lowering the placebo effect in a clinical study by including an additional group of study participants which are administered an Acebo.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Certain terminology is used in the following description for convenience only and is not limiting. The term “a,” as used in the specification, means “at least one.”

“About” as used herein when referring to a measurable value such as an amount, a temporal duration, and the like, is meant to encompass variations of ±20%, ±10%, ±5%, ±1%, or ±0.1% from the specified value, as such variations are appropriate.

Throughout this disclosure, various aspects of the present invention can be presented in a range format. It should be understood that the description in range format is merely for convenience and brevity and should not be construed as an inflexible limitation on the scope of the present invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Acebo” and “Acebo substance” as used herein refers to a substance that has at least one known effect which can worsen the disease or disorder or symptom of the disease or disorder that is being studied in the clinical trial, or can cause one of the primary or secondary subsymptoms of the disease or disorder that is being studied in the clinical trial, or can cause a related or unrelated effect of the disease or disorder that is being studied in the clinical trial.

“Arm” as used herein refers to a group of subjects in a clinical trial.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. A disease or disorder is “worsened” if the severity, intensity or impairment of a symptom of the disease or disorder, or the frequency with which such a symptom is experienced by a subject, or both, is increased.

The terms “subject,” “individual,” “participant,” and the like are used interchangeably herein, and refer to any animal amenable to the methods described herein. In certain non-limiting embodiments, the subject, individual or participant is a human.

“Medication,” “medicine,” and “drug” and “product” and “treatment” are used interchangeably to refer to a substance or intervention that treats or prevents or alleviates the symptoms of a disease.

“Responders” as used herein refers to those study participants who respond to a treatment, a placebo, or an Acebo.

“Partial responders” as used herein refers to those study participants who respond in a limited way or to a limited degree to a treatment, a placebo, or an Acebo.

“Non-responders” as used herein refers to those study participants who do not respond to a treatment, a placebo, or an Acebo.

Furthermore, the described features, advantages and characteristics of the embodiments of the present invention may be combined in any suitable manner in one or more embodiments. One skilled in the relevant art will recognize, in light of the description herein, that the present invention can be practiced without one or more of the specific features or advantages of a particular embodiment. In other instances, additional features and advantages may be recognized in certain embodiments that may not be present in all embodiments of the present invention.

### DESCRIPTION

The invention relates to the unexpected discovery of a novel approach to lowering the placebo effect in a clinical study by including an additional group of study participants which are administered an Acebo. In one aspect, the study participants are informed that they may be receiving a substance that has at least one known effect which can worsen the disease or disorder that is being studied in the clinical trial, i.e., an Acebo. While not being constrained by theory, it is suggested that lowering of the expectations of all subjects in the CT may reduce the overall placebo effect for that given CT (referred to as the “Acebo effect”). While not being constrained by theory, it is suggested that lowering of the expectations of all subjects in the CT may reduce the overall effect for that given CT (referred to as the “Acebo phenomenon”).

In one aspect, the invention relates to a method for lowering a placebo effect in a clinical trial by adding an additional (Acebo) arm to the CT protocol. For example, a third arm comprising an Acebo-treated group would be added to a clinical trial protocol if there are already two arms in that CT; a fourth arm comprising an Acebo-treated group would be added to a clinical trial protocol if there were normally three arms in that CT, etc.

In a standard double-blind placebo-controlled CT, (“DBPCCT”), for instance, comprising two arms, one arm may consist of subjects who receive an investigational medication (also referred to herein as “test substance”) and one arm may consist of subjects who receive a placebo. If the study protocol in a standard double-blind, placebo-controlled study consists of three arms, the design can be as follows: one arm for subjects who receive an investigational medication; one arm for subjects who receive an active/comparator medication; and one arm for subjects who receive a placebo. Alternatively, in a standard double-blind placebo controlled clinical research trial with three arms, the three arms may include: one arm for subjects who receive one dose of an investigational medication; one arm for subjects who receive a different dose of the investigational medication; and one arm for subjects who receive a placebo.

In one aspect of the invention, a standard two arm, double-blind placebo-controlled CT comprising administering an investigational medication to a first group of subjects and administering a placebo to a second group of subject can be modified to include a third arm comprising administering an Acebo to a third group of subjects.

In another aspect of the invention, a three arm, double-blind placebo-controlled CT comprising administering an investigational medication to a first group of subjects, administering a comparator medication or a different dose of the investigational medication to a second group of subjects, and administering a placebo to a third group of subject can be modified to include a fourth arm comprising administering an Acebo to a fourth group of subjects.

In one aspect, the invention relates to a method comprising N+X arms, wherein N refers to groups of subjects who are not administered an Acebo, for example, a group of subjects receiving: an investigational medication, a different dose of the investigational medicine, a comparator medicine, or a placebo, and X refers to one or more groups of subjects who are administered one or more Acebo substances and/or one or more doses of an Acebo. For example, X can comprise a first group receiving a first Acebo, a second group receiving a second Acebo or a different dose of the first Acebo, etc.

The Acebo effect may also measure nonresponders. For example, subjects who received an investigational medicine and improved significantly (i.e., responders) in Phase I then become subjects in Phase II. In Phase II, subjects are randomized to receive the investigational medication, placebo or Acebo and analysis of responders, partial responders, nonresponders, etc. is analyzed as described herein to determine the Acebo effect.

In certain aspects, the percentage of participants in the Acebo arm can be between 0% to 90%, 5% to 50%, or 10% to 33% of the number of participants in the clinical trial.

An Acebo arm can be added to any research study or clinical trial, including but not limited to medical, physical, psychiatric, psychological, advertising, marketing, commercial, biological, veterinarian, children, adolescent, adults, geriatric, digital, auditory, verbal, national, international, cross cultural, engineering, aesthetic, cosmetic, gender specific, culture specific, ethnicity specific, pain, neurologic, rheumatologic, cardiac, pulmonary, surgical, ophthalmologic, anesthetic, gastrointestinal, rehabilitative, addiction-related, otolaryngeal, dental, vascular, orthopedic, bariatric, obesity, urological, endocrinological, gynecological, obstetrical, podiatric, chiropractic, dermatological, oncological, infection-related, esthetic, any field involving human objective or subjective measurements, etc., or a combination of any of these, in placebo controlled trials, non-placebo-controlled trials, or other trials not specifically enumerated, etc.

An Acebo may be selected for administration based on its ability to mimic at times, worsen at times or cause one or more of the symptoms of a disease or disorder for which the investigational medicine is being tested. If there is no Acebo which mimics or worsens or causes one or more of the symptoms of a disease or disorder for which the investigational medicine is being tested, selection of the Acebo to be used in the methods may be based on its ability to cause one of the primary or secondary sub-symptoms of the disease or disorder or cause a related or unrelated effect of the disease or disorder. Other factors which can be considered during Acebo selection include: state of health to be achieved and symptom(s) of the subjects being studied. Other factors which can be considered during Acebo selection include previous exposure or previous lack of exposure to the Acebo. The Acebo can be, but is not limited to, foul tasting substance, an FDA-approved drug, an FDA-approved treatment, an FDA-approved product, an FDA-approved method, an FDA approved procedure, an FDA-approved over the counter medication, a food, an FDA approved medical food, a governmental approved drug, treatment, product, method, procedure, over the counter medication, or medical food, an unapproved drug, treatment, product, method, procedure, over the counter medication, or medical food, a plant-derived substance, an organic substance, a mineral, a non-organic substance, an artificial substance.

In another embodiment, the selection of the Acebo to be used in the methods may be based on its ability to cause a related disorder or symptom or an unrelated disorder or symptom.

In another embodiment the Acebo is added adjunctively to at least one of the arms of the study, i.e. a foul tasting substance, an FDA-approved over the counter medication, a food, an FDA-approved medical food, or a plant-derived substance. In one aspect, the Acebo is an FDA-approved medication. In another aspect, the Acebo is not an FDA-approved medication.

In one aspect, the present invention provides a method for lowering a placebo effect in a clinical trial comprising administering a test substance to a first group of a plurality of treatment groups of study participants, administering a placebo to a second group of said plurality of treatment groups of study participants, administering an Acebo to a subject in a third group of said plurality of treatment groups of study participants, wherein a participant in each of said treatment groups has been informed that he or she may be receiving a test substance, a placebo or an Acebo, determining whether each participant in each of said treatment groups is a responder, a partial responder or a non-responder; and comparing the percentage of responders or partial responders in the first (test) group to the percentage of responders or partial responders in the second (placebo) group. In another embodiment, the percentage of responders or partial responders in the first (test) group is compared to the percentage of responders or partial responders in the third (Acebo) group. In yet another embodiment, the percentage of responders or partial responders in the first (test) group is compared to the sum of the percentage of responders or partial responders in the second (placebo) group and the percentage of responders or partial responders in the third (Acebo) group.

In another aspect, the present invention provides a method for inducing an Acebo effect in a clinical trial comprising administering a test substance to a first group of a plurality of treatment groups of study participants and administering a substance that has at least one known effect which can worsen the disease or disorder that is being studied in the clinical trial (an Acebo) to a second group of said plurality of treatment groups of study participants, wherein each participant in each of said treatment groups has been informed that he or she may be receiving a test substance or an Acebo that has at least one known effect which can worsen the disease or disorder that is being studied in the clinical trial, determining whether each participant in each of said treatment groups is a responder a partial responder or a non-responder; and comparing the percentage of responders or partial responders or non-responders in the first (test) group to the percentage of responders or partial responders or non-responders in the second (Acebo) group.

In accordance with another exemplary embodiment, the present invention provides a method for lowering a placebo effect in a clinical trial comprising performing a first phase of testing, said first phase of testing including administering a test substance to a first group of a plurality of treatment groups of study participants, and administering a placebo to a remainder of said plurality of treatment groups of study participants, determining whether each participant in each of said treatment groups in said first phase is a responder or a non-responder; comparing the percentage of responders in the first (test) group of said first phase to the percentage of responders in the second (placebo) group of said first phase, and performing a second phase of testing, said second phase of testing including administering a test substance to a first group of a plurality of treatment groups of study participants, administering a placebo to a second group of said plurality of treatment groups of study participants, administering an Acebo to a third group of said plurality of treatment groups of study participants, wherein each participant in each of said treatment groups has been informed that he or she may be receiving a test substance, a placebo or an Acebo; determining whether each participant in each of said treatment groups in said second phase is a responder or a non-responder; comparing the percentage of responders in the first (test) group in said second phase to the percentage of responders in the second (placebo) group in said second phase, and analyzing data from said first phase of testing and data from said second phase of testing with a processor, wherein a greater difference between participants receiving the test substance and participants receiving the placebo in said second phase compared to the difference between participants receiving the test substance and participants receiving the placebo in said first phase indicates a lowering of said placebo effect. The methods described herein may further comprise determining whether each participant in each of said treatment groups is a partial responder and comparing the percentage of responders, partial responders and nonresponders.

Examples of theoretical statistics of how an Acebo could improve separation of drug from placebo are described as follows. In historical double-blind placebo controlled research clinical trial (DBPC RCT) comprising 300 subjects that on average improved 8 points when taking placebo and 300 subjects that on average improved 10 points when taking active drug, the DBPC RCT would show a 25% improvement on active medication compared with placebo.

Using the Acebo method described herein, if 300 subjects on placebo improved an average by 4 points while 300 subjects on active medication improved by 6 points, this would be 50% more of an improvement on active medication compared with placebo. This increased improvement is more likely to achieve the primary efficacy measure.

Results could also be viewed as percent responders on placebo versus percent responders on active medication. In the standard DBPCCT, if 40% of the placebo subjects were responders and 60% of the active medication subjects were responders, there would be 50% more responders on active medication than on placebo. If in that same clinical trial, an Acebo was used to decrease the placebo response and the number of placebo subjects responding was reduced to 30% while the number of active medication responders was reduced, in a similar number of subjects, to 50%, the study would demonstrate 66.6% more responders on active medication compared with placebo.

Determining the effect of an investigational medicine is an objective, fluid element based on several factors such as the disease or disorder being studied and the study conditions. For example, effect may be measured as percent improvement, absolute score, point change, etc. In a clinical study of depression, the effect may be measured using different rating scales, for example, the Hamilton Rating Scale for Depression (HRSD or HAM-D) or the Montgomery-Asberg Depression Rating Scale (MADRS). In certain investigational studies, effect can be measured as response, partial response or non-response whereas other investigational studies can measure effect in terms of remission, incomplete remission, partial remission or non-remission. Further, the distinction of the level of effect depends on or is set by the investigational study protocol. For example, an average positive change of 50% in one study may be considered a response or remission whereas it may be considered a partial response or partial remission in another study.

In one embodiment, the clinical trial is applied to any type of disease or disorder or condition, including but not limited to anxiety, depression, mania, hypertension, hypotension, pain, inflammation, gastroesophageal reflux disease (GERD), Alzheimer's disease, diabetes, constipation, Parkinson's disease, GI bleeding, gout, memory loss and dementia.

The disease or disorder can be any disease or disorder. In certain embodiments, the disease or disorder is any disease or disorder recognized in the International Statistical Classification of Diseases and Related Health Problems (ICD).

In one embodiment, the Acebo can increase mania (excessive elation and/or irritability). Acebos which can increase mania include but are not limited to: corticosteroids (such as triamcinolone acetonide, fluticasone, hydrocortisone, prednisone, and triamcinolone), immunosuppressants (such as cyclosporine), dopamine promoters (such as carbidopa/levodopa), muscle relaxants (such as baclofen), antidepressants (for example, monoamine oxidase inhibitors (MAOIs) such as phenelzine and tranylcypromine; selective serotonin reuptake inhibitors (SSRIs) such as escitalopram, fluoxetine, and paroxetine; serotonin/norepinephrine reuptake inhibitors (SNRIs) such as desvenlafaxine, duloxetine, levomilnacipran, venlafaxine, and tricyclic antidepressants (such as nortriptyline)), stimulants (such as methylphenidate and amphetamines), hormones (such as levothyroxine), antibiotics (such as ciproflozacin and gentamicin), antimalarial drugs (such as chloroquine and mefloquine), and antineoplastic drugs (such as 5-fluorouracil and ifosfamide).

In one embodiment, the Acebo can increase depression. Acebos which can increase depression include but are not limited to barbiturates (such as phenobarbital and secobarbital), benzodiazepines (such as alprazolam, clonazepam, chlordiazepoxide, diazepam, flurazepam, lorazepam, and triazolam), isotretinoin, hormones (for example, estrogens and progestogens such as etonogestrel, drospirenone, levonorgestrel, ethinyl estradiol, norethindrone, norgestimate, norgestrel, and desogestrel), corticosteroids, opioids (such as codeine, morphine, aspirin/oxycodone, meperidine, and oxycodone), anticholinergics, high blood pressure medications (for example, calcium-channel blockers such as diltiazem, nifedipine, and verapamil, beta-blockers such as atenolol, carvedilol, and metoprolol, and alpha 2 agonists such as clonidine and guanfacine), interferon alpha, alcohol, anticonvulsants (such as ethosuximide and methsuximide), varenicline, acyclovir, Parkinson's medication, such as carbidopa-levodopa and carbidopa-levodopa-entacapone, dopamine agonists such as pramipexole and ropinorole, stimulants, anticonvulsants, proton pump inhibitors, H2 blockers, anticholinergic drugs used to treat stomach cramps and other GI disorders, and statins (such as atorvastatin, fluvastatin, pravastatin, and simvastatin).

In one embodiment, the Acebo can increase hypertension. Acebos which can increase hypertension include but are not limited to: acetaminophen, non-steroidal anti-inflammatory drugs (NSAIDs) (such as aspirin, meloxicam, ibuprofen and naproxen), indomethacin, piroxicam, decongestants (such as pseudoephedrine and phenylephrine), alcohol, amphetamines, 3,4-methylenedioxymethamphetamine (MDMA) and its derivatives, cocaine, caffeine, methylphenidate, antidepressants (such as venlafaxine, bupropion, desipramine, monoamine oxidase inhibitors, tricyclic antidepressants, and fluoxetine), corticosteroids, cyclosporine, tacrolimus, erythropoietin, migraine medications, and herbal supplements (such as arnica (Arnica montana), bitter orange (Citrus aurantium), ephedra (ma-huang), ginkgo (Ginkgo bilboa), ginseng (Panax quinquefolius and Panax ginseng), guarana (Paullinia cupana), licorice (Glycyrrhiza glabra), senna (Cassia senna), and St. John's wort (Hypericum perforatum)).

In one embodiment, the Acebo can increase anxiety. Acebos which can increase anxiety include but are not limited to: bronchodilators (such as albuterol, salmeterol, and theophylline), anti-hypertensives (such as methyldopa), hormones (for example, estrogens and progestogens such as etonogestrel, drospirenone, levonorgestrel, ethinyl estradiol, norethindrone, norgestimate, norgestrel, and desogestrel), stimulants (such as amphetamines, methylphenidate, cocaine, and caffeine), cognitive-enhancing medications (such as atomoxetine), steroids (such as cortisone, dexamethasone, and prednisone), thyroid medicines (such as levothyroxine), decongestants (such as pseudoephedrine and phenylephrine), phenytoin, levodopa, quinidine, and antidepressants (such as bupropion, fluoxetine, venlafaxine, and selective serotonin reuptake inhibitors (SSRIs) such as escitalopram, fluoxetine, and paroxetine).

In one embodiment, the Acebo can worsen diabetes. Acebos which can worsen diabetes include but are not limited to: glucocorticoids, thiazide diuretics, beta-blockers, niacin, and antipsychotic drugs (such as olanzapine, clozapine, aripiprazole, chlorpromazine, flufenazine, haloperidol, molindone, perphenazine, perphenazine and amitriptyline, risperidone, thioridazine, thiothixene, and atypical antipsychotics such as quetiapine, cariprazine, brexpiprazole, ziprasidone, and asenapine).

In one embodiment, the Acebo can worsen GERD. Acebos which can worsen GERD include but are not limited to: antibiotics (such as tetracycline), bisphosphonates (such as alendronate, ibandronate and risedronate), iron supplements, quinidine, pain relievers (such as ibuprofen, naproxen and aspirin), potassium supplements, anticholinergics (such as oxybutynin), tricyclic antidepressants (such as amitriptyline and doxepin), blood pressure medicines (such as calcium channel blockers and beta blockers), narcotics (opioids) (such as codeine, morphine, oxycontin and hydrocodone and acetaminophen), hormones (such as progesterone), asthma medicines (such as theophylline), sedatives (for example, benzodiazepines such as diazepam and temazepam), antidepressants (such as imipramine and amitriptyline), and anticholinergics (such as oxybutynin and promethazine).

In one embodiment, the Acebo can cause or worsen constipation. Acebos which can worsen constipation include but are not limited to: amitriptyline, doxepin HCl, imipramine, amitriptyline, trimipramine, doxepin, desipramine, nortriptyline, protriptyline, haloperidol, pianozide, risperidone, thiothixene, olanzapine, clozapine, chlorpromazine, thioridazine, calcium-channel blockers, clonidine, clonidine/chlorthalidone, disopyramide, bromocriptine, trihexyphenidyl, benztropine mesylate, biperiden, procyclidine, cholestyramine, sucralfate, ferrous gluconate, ferrous sulfate, hydrocodone bitartrate, chlorpheniramine polistirex, hydrocodone tartrate, homatropine methylbromide, sulindac, ketoprofen, loperamide hydrochloride, acetaminophen and codeine, fentanyl, hydrocodone, hydromorphone, meperidine, methadone, morphine/morphine sulfate, oxycodone, oxymorphone, propoxyphene, tramadol, and St. John's Wort.

In one embodiment, the Acebo can worsen movement symptoms of Parkinson's disease, including slowness, stiffness, tremor and dyskinesia. In one embodiment, the Acebo is selected from the group consisting of, but not limited to: aripiprazole, chlorpromazine, flufenazine, haloperidol, molindone, perphenazine, perphenazine and amitriptyline, risperidone, thioridazine, thiothixene, and atypical antipsychotics such as quetiapine, cariprazine, brexpiprazole, ziprasidone, asenapine, as well as the anti-nausea medicines to avoid metoclopramide, phenothiazine, promethazine.

In one embodiment, the Acebo can worsen GI ulcers and GI bleeding. In one embodiment, the Acebo is selected from the group consisting of, but not limited to: diflunisal, ibuprofen, naproxen, flurbiprofen, diclofenac-misoprostol, aspirin diclofenac, cefuroxime, cinoxacin, sulindac, oxaprozin, ketorolac, ketorolac tromethamine, acetaminophen, acetaminophen-aspirin-caffeine, piroxicam, ketoprofen, indomethacin, etodolac, meclofenamate, meloxicam, nabumetone, fenoprofen, bisoprolol fumerate, piroxicam, mefenamic acid, tolmetin, and acetaminophen-naproxen.

In one embodiment, the Acebo is a substance that can worsen cognitive symptoms, including but not limited to those that block the effects of acetylcholine, such as tolteridine, some antidepressants (especially the tricyclics such as amitriptyline), antipsychotics, cardiac medications, antispasmodics, antivertigo medications, and antiparkinsonian medications having anticholinergic effects, anxiety and insomnia medications, benzodiazepines, such as lorazepam or sleeping pills such as temazepam, corticosteroids, pain Medications, and chemotherapy.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, practice the claimed methods. The following working examples therefore, specifically point out the preferred embodiments of the present invention, and are not to be construed as limiting in any way the remainder of the disclosure.

### Example 1

A clinical trial is conducted to measure the efficacy of a new product as an adjunct for the treatment of depression in subjects already on an antidepressant. One thousand fifty (1050) subjects are enrolled. All participants are maintained on their present antidepressant. The present antidepressant is supplied by the clinical treatment team; subjects no longer need to get their own prescriptions filled. The investigational product is supplied by the investigational treatment team; both the present antidepressant and the investigational product are taken once daily.

The subjects are divided into two test phases, Phase I and Phase II. All subjects sign an informed consent document prior to beginning the clinical research trial.

All subjects in Phase I are informed of the following:
1. 250 of the subjects will be randomized to the new investigational medication, product X, and the known benefits and the known and potential side effects of Product X are disclosed.
2. 250 of the subjects will be blindly randomized to placebo and the known benefits and the known and potential side effects of Product X are disclosed.
Subjects will be told, “Either you will receive the investigational medication or placebo, a blank/empty/inert/“sugar pill.” We do not know if the investigational medication will make you the same, better or worse. The results from all the subjects on the investigational medication will be compared to the results from all of the subjects on the placebo. The statistics of the study do not care if you stay the same, get better or get worse. But, your safety comes first. Should you worsen during the course of the clinical trial, either we will ask you to stop participating in the clinical trial or you can withdraw consent and stop participating in the clinical trial at any time.”

All subjects in Phase II are informed of the following:

1. 250 of the subjects will be randomized by a blinded computer to the new investigational medication, product X, and the known benefits and the known and potential side effects of Product X are disclosed.
2. 250 of the subjects will be blindly randomized by a blinded computer to placebo and the known benefits and the known and potential side effects of Product X are disclosed.
3. 50 of the subjects will be blindly randomized by a blinded computer to the Acebo clonidine, a medication approved for both hypertension and attention-deficit/hyperactivity disorder that is known with absolute certainty to cause depression in nearly 3% of those who have taken clonidine and had side effects from it. Depression is considered a common side effect of clonidine occurring between 1% and 10% of the time (https://www.drugs.com/sfx/clonidine-side-effects.html). Among the 31,961 people reported to have side effects when taking clonidine, 932 people (2.92%) developed depression. All subjects are informed about this well-known side effect and that they might be receiving the Acebo.
Subjects who have previously taken clonidine, whether s/he had a side effect or not, will be placed by the computer in a group of people who instead will receive a very similar medication, also approved for both hypertension and attention-deficit/hyperactivity disorder, the alternative Acebo, guanfacine, which does not cause depression, but on average causes somnolence (a quality or state of being drowsy) in 34% of people, fatigue (tiredness) report in 16% of people, and lethargy (the quality or state of being drowsy and dull, listless and unenergetic, or indifferent and lazy; apathetic or sluggish inactivity) in 6% of people. All three of these symptoms are associated with low interest, low energy, and a sleep disturbance, all symptoms of a major depressive episode.
Subjects will be told, “Your safety comes first. You may receive the investigational medication, you may receive the placebo or you may receive one of two different Acebos. In this study you could stay the same, get better or get worse. The results from all the subjects on the investigational medication will be compared to the results from all of the subjects on the placebo, a blank/empty/inert/“sugar pill.” Both of the Acebos, one of which you could receive, have been proven to make some subjects worse. If you do receive the Acebo, your depression could get worse. The statistics of the study do not care if you stay the same, get better or get worse. Should you worsen during the course of the clinical trial, either we will ask you to stop participating in the clinical trial or you can withdraw consent and stop participating in the clinical trial at any time.”

Results: Prior to participation in both Phases I and II, the average MADRS is 35. In Phase I, subjects who receive the investigational medicine have an average MADRS of 15 and subjects who receive the placebo have an average MADRS of 20. In Phase II, subjects who receive the investigational medicine have an average MADRS of 20, subjects who receive the placebo have an average MADRS of 25 and subjects who receive the Acebo have an average MADRS of 26. Thus, inclusion of an Acebo arm in the protocol resulted in a lowering of the placebo effect.

### Example 2

A clinical trial is being conducted to measure the efficacy of a new product for the treatment of constipation in subjects already on an opioid pain medication. Nine hundred sixty (960) subjects are enrolled. All are maintained on their present pain medication regimen supplied by the clinical treatment team. The investigational product is supplied by the investigational treatment team as well. The opioid pain medication regimen continues as it was; the investigational product is taken either once or twice per day in a randomized manner.

All subjects sign an informed consent document prior to beginning the clinical research trial. They are all informed of the following:
1. 300 of the subjects will be randomized to the new investigational medication, product Y, once daily, and the known benefits and the known potential side effects of Product Y are disclosed. To keep the study blinded, subjects will take a pill twice a day, but one of their doses will be a placebo.
2. 300 of the subjects will be randomized to the new investigational medication, product Y, twice daily, and the known benefits and the known potential side effects of Product Y are disclosed.
3. 300 of the subjects will be randomized to placebo taken twice a day.
4. 30 of the subjects will be randomized to an Acebo, nortriptyline, a medication approved for depression that is known with absolute certainty to cause constipation 10% or more of the time ([https://www.drugs.com/sfx/nortriptyline-side-effects.html]).
Subjects who are randomized to receive nortriptyline and have previously taken nortriptyline, whether s/he had a side effect or not, will be placed in the group of subjects to receive the Acebo St. John's wort, an over the counter substance also commonly known to cause constipation ([https://www.drugs.com/cdi/st-john-s-wort.html]).

5. 30 of the subjects will be randomized to the Acebo, St. John's wort, an over the counter substance also commonly known to cause constipation ([https://www.drugs.com/cdi/st-john-s-wort.html]).
Subjects who are randomized to receive St. John's wort and have previously taken St. John's wort, whether s/he had a side effect or not, will be placed in the group of subjects to receive an Acebo, nortriptyline, a medication approved for depression that is known with absolute certainty to cause constipation 10% or more of the time ([https://www.drugs.com/sfx/nortriptyline-side-effects.html]).
Your safety comes first. Because you could receive an Acebo, you could get worse. Should you significantly worsen during the course of the clinical trial, either we will ask you to stop participating in the clinical trial or you can withdraw consent and stop participating in the clinical trial at any time.

Results: Subjects receiving the investigational medicine, once a day showed an average improvement of 40%. Subjects receiving the investigational medicine, twice a day, showed an average improvement of 48%. Subjects receiving a placebo showed an average improvement of 20%. Subjects receiving nortriptyline showed a worsening of 2%. Subjects receiving St. John's wort nortriptyline showed a worsening of 1%. The investigational medicine is an efficacious drug for reducing constipation caused by opioid pain medication. Taking the medicine twice a day improves response by 20% compared to taking the medicine once a day.

### Example 3

A clinical trial is being conducted to measure of the efficacy of two different doses of a new product for the treatment of constipation in subjects not on any adjunctive medication that could cause constipation. Nine hundred sixty (960) subjects are enrolled. The investigational product is supplied by the investigational treatment team. Subjects who have been on both of the potential Acebos, nortriptyline and St John's wort, will be excluded from this study.

All subjects sign an informed consent document prior to beginning the clinical research trial. They are all informed of the following:
1. 300 of the subjects will be randomized to the new investigational medication, product Y, once daily, and the known benefits and the known and potential side effects of Product Y are disclosed. To keep the study blinded, subjects will take a pill twice a day, but one of their doses will be a placebo.
2. 300 of the subjects will be randomized to the new investigational medication, product Y, twice daily, and the known benefits and the known and potential side effects of Product Y are disclosed.
3. 300 of the subjects will be randomized to placebo taken twice a day.
4. 30 of the subjects will be randomized to the Acebo, nortriptyline, a medication approved for depression that is known with absolute certainty to cause constipation 10% or more of the time ([https://www.drugs.com/sfx/nortriptyline-side-effects.html]) twice daily unless they took nortriptyline before.

Subjects who are randomized to receive nortriptyline and have previously taken nortriptyline, whether s/he had a side effect or not, will be placed in the group of subjects to receive St. John's wort, an over the counter substance also commonly known to cause constipation ([https://www.drugs.com/cdi/st-john-s-wort.html]) twice daily.

5. 30 of the subjects will be randomized to the Acebo, St. John's wort, an over the counter substance also commonly known to cause constipation ([https://www.drugs.com/cdi/st-john-s-wort.html]) twice daily unless they took St. John's wort before.
Subjects who were randomized to receive St. John's wort and had previously taken St. John's wort, whether s/he had a side effect or not, will be placed in the group of subjects to receive the Acebo, nortriptyline, a medication approved for depression that is known with absolute certainty to cause constipation 10% or more of the time ([https://www.drugs.com/sfx/nortriptyline-side-effects.html]).
Your safety comes first. Should your constipation significantly worsen during the course of the clinical trial, which is a definite possibility, given the known side effects of both nortriptyline and St. John's wort, either we will ask you to stop participating in the clinical trial or you can withdraw consent and stop participating in the clinical trial at any time.

Results: Subjects receiving the investigational medicine, once a day showed an average improvement of 10% in abdominal discomfort. Subjects receiving the investigational medicine, twice a day, showed an average improvement of 40% in abdominal discomfort. Subjects receiving a placebo showed an average improvement of 5% in abdominal discomfort. Subjects receiving nortriptyline showed a worsening of 1% in abdominal discomfort. Subjects receiving St. John's wort showed a worsening of 2% in abdominal discomfort. The investigational medicine is an efficacious drug for reducing constipation when taken twice daily; once daily Product Y did not separate from placebo.

### Example 4

A clinical trial is being conducted to measure of the efficacy of a new product for the treatment of anxiety in subjects not already on any medication known to cause anxiety. Nine hundred sixty (900) subjects are enrolled. The investigational product is supplied by the investigational treatment team. The investigational product is taken either once or twice per day in a randomized manner. Subjects with ADHD will be excluded. Subjects with Depression will be excluded. Subjects who have previously taken Adderall will be excluded.

All subjects sign an informed consent document prior to beginning the clinical research trial. They are all informed of the following:
1. 300 of the subjects will be randomized to the new investigational medication, product Z, at a known sub-therapeutic dose once daily. The known benefits and the known and potential side effects of Product Z are disclosed. To keep the study blinded, subjects will take a pill twice a day, one of the doses every day will be the Acebo, Adderall (amphetamine/dextroamphetamine). To additionally maintain the blind, the blister card will randomly vary the time of day Product Z and Adderall are taken. Some days Product Z will be the a.m. dose, some days Product Z will be the p.m. dose and vice versa.
2. 300 of the subjects will be randomized to the new investigational medication, product Z, at a presumably therapeutic dose, twice daily, and the known benefits and the known and potential side effects of Product Z are disclosed.
3. 300 of the subjects will be randomized to placebo taken twice a day.
Your safety comes first. Should your anxiety significantly worsen during the course of the clinical trial, which is a definite possibility, given the known side effects of Adderall, either we will ask you to stop participating in the clinical trial or you can withdraw consent and stop participating in the clinical trial at any time.

Results: Subjects in the first arm, receiving Product Z and the Acebo, Adderall, demonstrated a 30% improvement. Subjects receiving Product Z bid demonstrated a 40% improvement. Subjects receiving the placebo bid demonstrated a 29% improvement. The study was successful. Product Z's effect, when taken bid, as measured on the Hamilton Anxiety Scale, successfully separated from placebo and from once a day Product Z.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method for lowering a placebo effect in a clinical trial comprising:
administering a test substance to a participant in a first group of a plurality of treatment groups of study participants;
administering a placebo to a participant in second group of said plurality of treatment groups of study participants;
administering a substance that has at least one known effect which can worsen a disease or disorder that is being studied in the clinical trial, or can cause one of the primary or secondary subsymptoms of the disease or disorder that is being studied in the clinical trial, or can cause a related or unrelated effect of the disease or disorder that is being studied in the clinical trial (an Acebo) to a participant in a third group of said plurality of treatment groups of study participants;
determining whether each participant in each of said treatment groups is a responder, a partial responder or a non-responder, and
comparing the percentage of responders in the first group to the percentage of responders in the second group.

2. The method of claim 1, wherein said participant in each of said treatment groups has been informed that he or she may be receiving a test substance, a placebo or an Acebo.

3. The method of claim 1, further comprising determining the percentage of partial responders in each of said treatment groups and comparing the percentage of partial responders in the first group to the percentage of partial responders in the second group or the sum of the percentage of responders and percentage of partial responders in the first group to the sum of the percentage of responders and percentage of partial responders in the second group.

4. The method of claim 1, wherein the percentage of responders in the first group is compared to the percentage of responders in the third group.

5. The method of claim 1, wherein the percentage of responders in the first group is compared to the sum of the percentage of responders in the second and the percentage of responders in the third group.

6. The method of claim 1, wherein a percentage of participants in the third group is 0% to 90% of a total number of participants in the clinical trial.

7. The method of claim 1, wherein the disease or disorder is selected from the group consisting of anxiety, depression, mania, hypertension, hypotension, pain, inflammation, gastroesophageal reflux disease (GERD), Alzheimer's disease, diabetes, and constipation.

8. The method of claim 1, wherein the Acebo is a foul tasting substance, an FDA-approved drug, an FDA-approved treatment, an FDA-approved product, an FDA-approved method, an FDA approved procedure, an FDA-approved over the counter medication, a food, an FDA approved medical food, a governmental approved drug, treatment, product, method, procedure, over the counter medication, or medical food, an unapproved drug, treatment, product, method, procedure, over the counter medication, or medical food, a plant-derived substance, an organic substance, a mineral, a non-organic substance, or an artificial substance.

9. The method of claim 1, wherein the Acebo is selected from the group consisting of corticosteroids, immunosuppressants, dopamine promoters, muscle relaxants, antidepressants, stimulants, narcotics, anti-anxiety drugs, antibiotics, antimalarial drugs, hormones, antineoplastic drugs, bronchodilators, analgesics, anti-inflammatory drugs, cognition-enhancing drugs, antihypertensive drugs, anticholinergics, anticonvulsants, antipsychotic drugs, statins, vitamins, and herbal supplements.

10. The method of claim 1, wherein an Acebo is administered adjunctively to the participants in at least one of said treatment groups.

11. A method for inducing an Acebo effect in a clinical trial comprising:
administering a test substance to a first group of a plurality of treatment groups of study participants;
administering a substance that has at least one known effect which can worsen a disease or disorder that is being studied in the clinical trial, or can cause one of the primary or secondary subsymptoms of the disease or disorder that is being studied in the clinical trial, or can cause a related or unrelated effect of the disease or disorder that is being studied in the clinical trial (an Acebo) to a second group of said plurality of treatment groups of study participants;
determining whether each participant in each of said treatment groups is a responder or a non-responder, and
comparing the percentage of responders in the first (test) group to the percentage of responders in the second (Acebo) group.

12. The method of claim 11, wherein each participant in each of said treatment groups has been informed that he or she may be receiving a test substance or an Acebo.

13. The method of claim 11, wherein the disease or disorder is selected from the group consisting of anxiety, depression, mania, hypertension, hypotension, pain, inflammation, gastroesophageal reflux disease (GERD), Alzheimer's disease, diabetes, constipation Parkinson's disease, gastrointestinal bleeding, gout and memory loss or dementia.

14. The method of claim 11, wherein the Acebo is selected from the group consisting of corticosteroids, immunosuppressants, dopamine promoters, muscle relaxants, antidepressants, stimulants, narcotics, anti-anxiety drugs, antibiotics, antimalarial drugs, hormones, antineoplastic drugs, bronchodilators, analgesics, anti-inflammatory drugs, cognition-enhancing drugs, antihypertensive drugs, anticholinergics, anticonvulsants, antipsychotic drugs, statins, vitamins, and herbal supplements.

15. The method of claim 11, wherein the Acebo is foul tasting substance, an FDA-approved drug, an FDA-approved treatment, an FDA-approved product, an FDA-approved method, an FDA approved procedure, an FDA-approved over the counter medication, a food, an FDA approved medical food, a governmental approved drug, treatment, product, method, procedure, over the counter medication, or medical food, an unapproved drug, treatment, product, method, procedure, over the counter medication, or medical food, a plant-derived substance, an organic substance, a mineral, a non-organic substance, or an artificial substance.

16. The method of claim 11, wherein an Acebo is administered adjunctively to the participants in at least one of said treatment groups.

17. A method for lowering a placebo effect in a clinical trial comprising:
performing a first phase of testing, said first phase of testing including administering a test substance to a first group of a plurality of treatment groups of study participants, and administering a placebo to a remainder of said plurality of treatment groups of study participants;
determining whether each participant in each of said treatment groups in said first phase is a responder or a non-responder;
comparing the percentage of responders in the first group of said first phase to the percentage of responders in the second group of said first phase, and
performing a second phase of testing, said second phase of testing including administering a test substance to a first group of a plurality of treatment groups of study participants, administering a placebo to a second group of said plurality of treatment groups of study participants, and administering a substance that has at least one known effect which can worsen a disease or disorder that is being studied in the clinical trial, or can cause one of the primary or secondary subsymptoms of the disease or disorder that is being studied in the clinical trial, or can cause a related or unrelated effect of the disease or disorder that is being studied in the clinical trial (an Acebo) to a third group of said plurality of treatment groups of study participants;
determining whether each participant in each of said treatment groups in said second phase is a responder or a non-responder, and
comparing the percentage of responders in the first group in said second phase to the percentage of responders in the second group in said second phase, and analyzing data from said first phase of testing and data from said second phase of testing with a processor,
wherein a greater difference between participants receiving the test substance and participants receiving the placebo in said second phase compared to the difference between participants receiving the test substance and participants receiving the placebo in said first phase indicates a lowering of said placebo effect.

18. The method of claim 17, wherein each participant in each of said treatment groups has been informed that he or she may be receiving a test substance, a placebo or an Acebo.

19. The method of claim 17, further comprising determining the percentage of partial responders in each of said treatment groups and comparing the percentage of partial responders in said first group of said first phase to the percentage of partial responders in said second group of said first phase and comparing the percentage of partial responders in said first group of said second phase to the percentage of partial responders in said second group of said second phase.

20. The method of claim 17, further comprising determining the percentage of partial responders in each of said treatment groups and comparing the sum of the percentage of partial responders and responders in said first group of said first phase to the sum of the percentage of partial responders and responders in said second group of said second phase.

21. The method of claim 17, wherein the disease or disorder is selected from the group consisting of anxiety, depression, mania, hypertension, hypotension, pain, inflammation, gastroesophageal reflux disease (GERD), Alzheimer's disease, diabetes, constipation. Parkinson's disease, gastrointestinal bleeding, gout and memory loss or dementia.

22. The method of claim 17, wherein an Acebo is selected from the group consisting of corticosteroids, immunosuppressants, dopamine promoters, muscle relaxants, antidepressants, stimulants, narcotics, anti-anxiety drugs, antibiotics, antimalarial drugs, hormones, antineoplastic drugs, bronchodilators, analgesics, anti-inflammatory drugs, cognition-enhancing drugs, antihypertensive drugs, anticholinergics, anticonvulsants, antipsychotic drugs, statins, vitamins, and herbal supplements.

23. The method of claim 17, wherein the Acebo is a foul tasting substance, an FDA-approved drug, an FDA-approved treatment, an FDA-approved product, an FDA-approved method, an FDA approved procedure, an FDA-approved over the counter medication, a food, an FDA approved medical food, a governmental approved drug, treatment, product, method, procedure, over the counter medication, or medical food, an unapproved drug, treatment, product, method, procedure, over the counter medication, or medical food, a plant-derived substance, an organic substance, a mineral, a non-organic substance, or an artificial substance.

24. The method of claim 17, wherein the Acebo is administered adjunctively to the participants in at least one of said treatment groups.

25. A method for lowering a placebo effect in a clinical trial comprising:
administering a test substance to a participant in a first group of a plurality of treatment groups of study participants;
administering a substance that has at least one known effect which can worsen a disease or disorder that is being studied in the clinical trial, or can cause one of the primary or secondary subsymptoms of the disease or disorder that is being studied in the clinical trial, or can cause a related or unrelated effect of the disease or disorder that is being studied in the clinical trial (an Acebo) to a participant in a second group of said plurality of treatment groups of study participants;
determining whether each participant in each of said treatment groups is a responder or a non-responder, and
comparing the percentage of responders in the first group to the percentage of responders in the second group.

26. A method for lowering a placebo effect in a clinical trial comprising:
administering a test substance to a participant in a first group of a plurality of treatment groups of study participants;
administering a substance that has at least one known effect which can worsen a disease or disorder that is being studied in the clinical trial, or can cause one of the primary or secondary subsymptoms of the disease or disorder that is being studied in the clinical trial, or can cause a related or unrelated effect of the disease or disorder that is being studied in the clinical trial (an Acebo) to a participant in a second group of said plurality of treatment groups of study participants;
determining whether each participant in each of said treatment groups is a responder, a partial responder or a non-responder, and
comparing the percentage of responders and partial responders in the first group to the percentage of responders and partial responders in the second group.
